(19) United States
(12) Patent Application Publication
(10) Pub. No.: US 2004/0219115 A1
(43) Pub. Date: Nov. 4, 2004

(54) Cosmetic composition

(75) Inventors: Mridula Kini, Maharastra (IN); Lalitagauri Rajwade, Maharastra (IN); Pushker Sona, Bangkok (TH); Ramesh Surianarayanan, Maharastra (IN)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/767,091

(22) Filed: Jan. 29, 2004

(30) Foreign Application Priority Data
Jan. 31, 2003 (IN) 128/MUM/03

(51) Int. Cl. A61K 7/135

(52) U.S. Cl. 424/62

(57) ABSTRACT

A skin lightening composition comprising;

a) 0.05 to 10% by weight of one of more of vitamin B3 compounds or its derivatives thereof;

b) 0.01 to 10% by weight of allantoin; and

c) a cosmetically acceptable vehicle or 15% to 85% by weight detergent active.

The present invention relates to synergistic cosmetic compositions for lightening of skin colour. In particular, the composition of the invention relates to compositions for topical application to human skin to provide for lightening of skin colour.

Conventional skin lightening compositions are based on sunscreens which maintain skin colour against ultraviolet radiation and are based on materials which absorb these incident rays which are known to cause tanning of skin. Other approaches include use of skin lightening agents which are believed to control dispersion of melanosomes or inhibit tyrosinase. These skin-lightening agents include niacinamide, carboxylic acids like azelaic acid and kojic acid, plant extracts and hydroquinone etc. Niacinamide, which is a vitamin B3 compound, is one such widely used skin lightening agent in compositions for topical application. Skin lightening agents, along with sunscreen compounds, both organic and inorganic in nature, both naturally occurring and synthetically prepared, have been combined in skin lightening compositions to provide synergistic benefits.

EP0396422 (Unilever, 1990) discloses a skin lightening composition, comprising niacinamide, Parsol MCX and Parsol 1789, UV-B and UV-A sunscreens, as well as silicone oil in the skin lightening composition. The composition gives enhanced skin lightening.

U.S. Pat. No. 6,444,647 and U.S. Pat. No. 6,492,326 (both by Proctor & Gamble, 2002) disclose skin care compositions containing combinations of skin care actives. Both these compositions act to synergistically regulate (prophylactically and/or therapeutically) visible and/or tactile discontinuities in mammalian skin. These include fine lines, wrinkles, enlarged pores, roughness, dryness, and other skin texture discontinuities, e.g., they reduce or efface the visibility of fine lines, wrinkles, and other forms of uneven or rough surface texture associated with aged or photodamaged skin.

U.S. Pat. No. 6,444,647 discloses a dual acting system, particularly a vitamin B3 compound in combination with Farnesol and/or phytantriol. The above patent also claims compositions that provide enhanced skin care, that in addition to the above essential ingredients comprise an additional skin care active selected from the group consisting of allantoin, retinyl propionate, tocopherol, tocopherol esters, peptides, peptide derivatives, phytosterol, isoflavone, panthenol, bisabolol, salicylic acid, and mixtures thereof.

U.S. Pat. No. 6,492,326 discloses a skin care composition comprising (a) a safe and effective amount of a peptide active selected from the group consisting of pentapeptides, derivatives of pentapeptides and mixtures thereof, along with (b) a safe and effective amount of at least one additional skin care active selected from the group consisting of desquamatory actives, anti-acne actives, vitamin B3 compounds, retinoids, di-, tri-, and tetra- peptides and derivatives thereof, hydroxy acids, radical scavengers, chelators, anti-inflammatory agents, topical anaesthetics, tanning actives, skin lightening agents, anti-cellulite agents, flavonoids, antimicrobial actives, skin healing agents, antifungal actives, farnesol, phytantriol, allantoin, glucosamine, and mixtures thereof; and c) a dermatologically acceptable carrier.

WO98/34591 (Proctor & Gamble, 1998) describes a method of lightening hyperpigmented regions of skin, comprising topically applying to the skin a safe and effective amount of a composition comprising (a) an active effective for lightening hyperpigmented regions of skin and (b) a topical carrier, the active consisting essentially of tocopherol sorbate. In addition the composition further comprises a compound selected from the group consisting of anti-inflammatory agents, anti-oxidants/radical scavengers, retinoids, niacinamide and combinations thereof.

This patent also claims a composition suitable for lightening mammalian skin comprising: (a) tocopherol sorbate; (b) an anti-inflammatory agent; preferably the anti-inflammatory agent is selected from bisabolol, chamomile extract, compounds of the Licorice (Glycyrrhiza glabra) family and derivatives thereof, panthenol, methyl salicylate, aloe, allantoin and mixtures thereof; (c) an anti-oxidant/radical scavenger; preferably the anti-oxidant/radical scavenger is selected from ascorbic acid derivatives, more preferably wherein the anti-oxidant/radical scavenger is magnesium ascorbyl phosphate; (d) a retinoid; preferably the retinoid is selected from retinol palmitate, retinol acetate, retinol propionate, retinol and mixtures thereof, and (e) a topical carrier.

Allantoin has been used in skin creams, and is used as an anti-inflammatory agent, or as a skin-soothing or a healing agent.

None of the prior art teaches that a combination of a vitamin B3 compound and an anti-inflammatory agent like allantoin leads to synergistic skin lightening benefits.

The applicants have now surprisingly found that a composition comprising a combination of allantoin and a vitamin B3 compound or its derivatives leads to synergistic benefits in skin lightening efficacy. The compositions are useful both for normal skin, as well as skin damaged by ultra violet radiation. The compositions are also useful in treating freckles, hyper-pigmented skin, blotchy skin, age spots etc.

It is thus an object of the present invention to provide for a composition which may demonstrate synergy that has enhanced skin lightening benefits.

It is a further object of the invention to provide for a highly effective skin lightening composition that is safe to use, and utilises widely and cheaply available ingredients.

It is yet another object of the present invention to provide for a skin lightening composition that has synergistic benefits in skin lightening, and additionally provides anti-inflammatory and skin soothing benefits.

According to a first aspect of the invention, there is provided a synergistic skin lightening composition comprising:

a) 0.05% to 10% by weight of one or more of vitamin B3 compound or its derivative thereof;

b) 0.01% to 10% by weight of allantoin; and

c) a cosmetically acceptable vehicle, or 15%-85% of a detergent active.

According to a preferred aspect of the invention, there is provided a skin lightening composition comprising:

a) 0.1% to 10% by weight of niacin, niacinamide, or a precursor thereof;

b) 0.01% to 10% by weight of allantoin; and

c) a cosmetically acceptable vehicle, or 15%-85% detergent active.

According to another preferred aspect of the invention, there is provided a skin lightening composition comprising:

a) 0.2% to 5% by weight of niacinamide or a precursor thereof;

b) 0.05% to 5% by weight of allantoin;

c) 0.1% to 10% by weight of one or more sunscreens; and

d) a cosmetically acceptable vehicle, or 15%-85% detergent active.

It is particularly preferred that the sunscreen is chosen from 2-ethyl hexyl-p-methoxycinnamate, 4,4′-t-butyl methoxydibenzoyl-methane, titanium dioxide or mixtures thereof.

All parts herein are by weight unless otherwise specified.

The present invention pertains to synergistic skin lightening compositions comprising one or more of a vitamin B3 compound, preferably niacinamide, along with allantoin and optionally one or more sunscreens.

The compositions of the invention can be leave on products in the form of lotions, creams, gels, mousses etc., or in the form of rinse off products such as soap bars, detergent powders, flakes, face wash products, body wash products, etc.

It is an essential feature of the invention that one or more of vitamin B3 compound or its derivatives be present in the skin lightening composition of the invention.

Vitamin B3 compounds that can be used as per the invention include but are not limited to niacin, niacinamide, nicotinyl alcohol, and derivatives and salts of of these compounds. Possible derivatives of the vitamin B3 compounds include nicotinic acid esters, nicotinyl alcohol esters of carboxylic acids, niacinamide N-oxide, nicotinyl amino acids and nicotinic acid n-oxide.

Preferred vitamin B3 compounds as per this invention are niacin and niacinamide, and the particularly preferred vitamin B3 compound is niacinamide.

Vitamin B3 is typically incorporated at a level of 0.05% to 10% by weight of the composition, and is preferably present in an amount from 0.1% to 10% by weight of the composition. A highly preferred amount of vitamin B3 compound is from 0.2% to 5% by weight of the composition.

Other vitamins which act as skin lightening ingredients can be advantageously included in the composition to provide for additional skin lightening effects. These include vitamin B6, vitamin C, vitamin A or their precursors. Mixtures of the vitamins can also be employed in the composition of the invention. Especially preferred additional vitamin is vitamin B6.

It is an essential feature of the invention that allantoin be present in the skin lightening composition of the invention.

Allantoin is usually available as a white, odourless, crystalline powder when isolated. Allantoin has the chemical name of (2,5 dioxo-4-imidazolidinyl) urea glyoxyldiureide or urea(2,5-dioxo-4-imidazolidinyl)-5-ureidohydantoin, and is available under the trade names of Allantoin Powder (Rona/EM Industries), Chemie Linz Allantoin (DSM Fine Chemicals), Fancol TOIN (Fanning), and from a host of other suppliers. Allantoin has been termed as a counter irritant that helps alleviate the untoward skin irritation effects of certain cosmetic ingredients such as soap and detergent surfactants, oils, and acidic and alkaline materials.

Allantoin has been incorporated in skin creams among other applications for its anti-inflammatory and skin-soothing and healing properties. Allantoin, as per this invention, is incorporated at 0.01% to 10% by weight of the composition, preferably in an amount of from 0.05% to 5% by weight of the composition, more preferably in an amount of from 0.05% to 2% by weight of the composition.

The composition of the invention also preferably includes an effective amount of a sunscreen or sun-block agent to enhance synergistically the benefit of the composition in providing for skin lightening. Organic and inorganic sunscreens/sun-blocks may be suitably employed in the composition.

Ultraviolet light is a predominant cause of skin darkening. Thus, for purposes of skin lightening, compositions comprising UVA and/or UVB sunscreen are desirable.

A wide variety of conventional sunscreen agents are suitable for use in combination with the essential ingredients of this invention. Suitable sunscreen agents include p-aminobenzoic acid, its salts and its derivatives (e.g. ethyl, isobutyl, glyceryl esters; p-dimethylaminobenzoic acid); anthranilates (e.g., o-aminobenzoates; methyl, menthyl, phenyl, benzyl, phenylethyl, linalyl, terpinyl, and cyclohexenyl esters); salicylates (e.g. amyl, phenyl, benzyl, menthyl, glyceryl, and dipropyleneglycol esters); cinnamic acid derivatives (e.g. menthyl and benzyl esters, a-phenyl cinnamonitrile; butyl cinnamoyl pyruvate); dihydroxycinnamic acid derivatives (e.g. umbelliferone, methylumbelliferone, methylaceto-umbelliferone); trihydroxycinnamic acid derivatives (e.g. esculetin, methylesculetin, daphnetin, and the glucosides, esculin and daphnin); hydrocarbons (e.g. diphenylbutadiene, stilbene); dibenzalacetone and benzalacetophenone; naphthol-sulfonates (e.g. sodium salts of 2-naphthol-3,6-disulfonic and of 2-naphthol-6,8-disulfonic acids); di-hydroxy-naphthoic acid and its salts; o- and p-hydroxybiphenyldisulfonates; coumarin derivatives (e.g. 7-hydroxy, 7-methyl, 3-phenyl); diazoles (e.g. 2-acetyl-3-bromoindazole, phenyl benzoxazole, methyl naphthoxazole, various aryl benzothiazoles); quinine salts (e.g. bisulfate, sulfate, chloride, oleate, and tannate); quinoline derivatives (e.g. 8-hydroxyquinoline salts, 2-phenylquinoline); hydroxy- or methoxy-substituted benzophenones; uric and vilouric acids; tannic acid and its derivatives (e.g., hexaethylether); (butyl carbotol) (6-propyl piperonyl) ether; hydroquinone; benzophenones e.g. oxy-benzene, sulisobenzone, dioxybenzone, benzoresorcinol, 2,2′,4,4′-tetrahydroxybenzophenone, 2,2′-dihydroxy-4,4′-dimethylbenzophenone, octabenzone; 4-isopropyldibenzoylmethane; butyl-methoxydibenzoylmethane; etocrylene; and 4-isopropyl-di-benzoylmethane.

Of these, 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl-p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, and mixtures of these compounds are preferred.

More preferred sunscreens useful in the compositions useful in the subject invention are 2-ethylhexyl-p-methoxycinnamate, butylmethoxydibenzoylmethane, 2-hydroxy-4-methoxybenzophenone, octyldimethyl-p-aminobenzoic acid and mixtures thereof.

Also particularly useful in the compositions are sunscreens such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991, both of which are incorporated herein by reference. The sunscreen agents disclosed therein have, in a single molecule, two distinct chromophore moieties that exhibit different ultra-violet radiation absorption spectra. One of the chromophore moieties absorbs predominantly in the UVB radiation range, and the other absorbs strongly in the UVA radiation range.

A safe and effective amount of sunscreen may be used in the compositions useful in the subject invention. The composition preferably comprises from about 0.1% to about 10%, more preferably from about 0.1% to about 5%, of a sunscreen agent, which amounts may be wholly either organic or inorganic sunscreen.

Useful inorganic sun-blocks are also preferably used in the present invention. These include, for example, zinc oxide iron oxide, silica such as fumed silica, and titanium dioxide.

Ultrafine titanium dioxide in either of its two forms, namely water-dispersible titanium dioxide and oil-dispersible titanium dioxide, is especially suitable for the invention. Water-dispersible titanium dioxide is ultra-fine titanium dioxide, the particles of which are non-coated or which are coated with a material to impart a hydrophilic surface property to the particles. Examples of such materials include aluminium oxide and aluminium silicate.

Oil-dispersible titanium dioxide is ultrafine titanium dioxide, the particles of which exhibit a hydrophobic surface property, and which, for this purpose, can be coated with metal soaps such as aluminium stearate, aluminium laurate or zinc stearate, or with organosilicone compounds.

By “ultrafine titanium dioxide” is meant particles of titanium dioxide having an average particle size of less than 100 nm, preferably 70 nm or less, more preferably from 10 to 40 nm and most preferably from 15 to 25 nm.

By topical application to the skin of a mixture of both water-dispersible ultrafine titanium dioxide and oil-dispersible ultrafine titanium dioxide, synergistically enhanced protection of the skin against the harmful effects of both UV-A and UV-B rays is achievable.

Ultrafine titanium dioxide is the preferred inorganic sun-block agent as per this invention. The total amount of sun block that is preferably incorporated in the composition according to the invention is from 0.1% to 5% by weight of the composition.

The composition according to the invention may also comprise a cosmetically acceptable vehicle to act as a diluant, dispersant or carrier for other materials present in the composition, so as to facilitate their distribution when the composition is applied to the skin.

Suitable vehicles other than water can include liquid or solid emollients, solvents, humectants, thickeners and powders. Examples of each of these types of vehicle, which can be used singly or as mixtures of one or more vehicles, are as follows:

Emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate;

Propellants, such as propane, butane, isobutane, dimethyl ether, carbon dioxide, nitrous oxide;

Solvents, such as ethyl alcohol, isopropanol, acetone, ethylene glycol monoethyl ether, diethylene glycol monobutyl ether, diethylene glycol monoethyl ether;

Powders, such as chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate.

The cosmetically acceptable vehicle will usually form from 10% to 99.9%, preferably from 50% to 99% by weight of the emulsion, and can, in the absence of other cosmetic adjuncts, form the balance of the composition.

Other skin lightening actives known in the art can also be employed in the invention. Non-limiting examples of skin lightening actives useful herein include adapalene, aloe extract, ammonium lactate, anethole derivatives, apple extract, arbutin, azelaic acid, bamboo extract, bearberry extract, bletilla tuber, bupleurum falcatum extract, burnet extract, butyl hydroxy anisole, butyl hydroxy toluene, citrate esters, Chuanxiong, Dang-Gui, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, FADEOUT (available from Pentapharm), Fangfeng, fennel extract, ganoderma extract, gaoben, GATULINE WHITENING (available from Gattlefosse), genistic acid and its derivatives, glabridin and its derivatives, gluco pyranosyl-1-ascorbate, gluconic acid, glycolic acid, green tea extract, 4-Hydroxy-5-methyl-3 [2H]-furanone, hydroquinone, 4 hydroxyanisole and its derivatives, 4-hydroxy benzoic acid derivatives, hydroxycaprylic acid, inositol ascorbate, kojic acid, lactic acid, lemon extract, linoleic acid, magnesium ascorbyl phosphate, MELAWHITE (available from Pentapharm), morus alba extract, mulberry root extract, 5-octanoyl salicylic acid, parsley extract, phellinus linteus extract, pyrogallol derivatives, 2,4 resorcinol derivatives, 3,5 resorcinol derivatives, rose fruit extract, salicylic acid, Song-Yi extract, 3,4,5 trihydroxybenzyl derivatives, tranexamic acid and mixtures thereof.

It is also possible to provide for the skin lightening composition of the invention is the form of a personal wash formulation, for example a soap bar.

The term total fatty matter, usually abbreviated to TFM, is used to denote the percentage by weight of fatty acid and triglyceride residues present, without taking into account the accompanying cations.

For soap having 18 carbon atoms, an accompanying sodium cation will generally amount to about 8% by weight. Other cations may be employed as desired for example zinc, potassium, magnesium, alkyl ammonium and aluminium.

The term soap denotes salts of carboxylic fatty acids. The soap may typically be derived from any of the triglycerides conventionally used in soap manufacture—consequently the carboxylate anions in the soap may contain from 8 to 22 carbon atoms.

The soap may be obtained by saponifying a fat and/or a fatty acid. The fats or oils generally used in soap manufacture may be such as tallow, tallow stearines, palm oil, palm stearines, soya bean oil, fish oil, caster oil, rice bran oil, sunflower oil, coconut oil, babassu oil, palm kernel oil, and others. In the above process the fatty acids are derived from oils/fats selected from coconut, rice bran, groundnut, tallow, palm, palm kernel, cotton seed, soya bean, castor etc. The fatty acid soaps can also be synthetically prepared (e.g. by the oxidation of petroleum or by the hydrogenation of carbon monoxide by the Fischer-Tropsch process). Resin acids, such as those present in tall oil, may be used. Naphthenic acids are also suitable.

Tallow fatty acids can be derived from various animal sources and generally comprise about 1% to 8% myristic acid, about 21-32% palmitic acid, about 14-31% stearic acid, about 0-4% palmitoleic acid, about 36-50% oleic acid and about 0-5% linoleic acid. A typical distribution is 2.5% myristic acid, 29% palmitic acid, 23% stearic acid, 2% palmitoleic acid, 41.5% oleic acid, and 3% linoleic acid. Other similar mixtures, such as those from palm oil and those derived from various animal tallow and lard are also included.

Coconut oil refers to fatty acid mixtures having an approximate carbon chain length distribution of 8% C₈, 7% C₁₀, 48% C₁₂, 17% C₁₄, 8% C₁₆, 2% C₁₈, 7% oleic and 2% linoleic acids (the first six fatty acids listed being saturated). Other sources having similar carbon chain length distributions, such as palm kernel oil and babassu kernel oil, are included within the term coconut oil.

Other detergent actives like synthetic anionic surfactants, cationic surfactants, amphoteric surfactants, zwitterionic surfactants or their mixtures thereof may also be present in the composition. Such actives are disclosed in standard detergent textbooks for example “Surface Active Agents”, Volume I by Schwartz and Perry and “Surface Active Agents and Detergents”, Volume II by Schwartz, Perry and Berch.

The compositions of the present invention can comprise a wide range of other optional cosmetic components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include antioxidants, binders, biological additives, buffering agents, colorants, thickeners, polymers, astringents, fragrance, humectants, opacifying agents, conditioners, exfoliating agents, pH adjusters, preservatives, natural extracts, essential oils, skin sensates, skin soothing agents, and skin healing agents.

The invention is now further described by way of the following non-limiting examples.

### EXAMPLES

Formulations in the form of creams were prepared using the ingredients as listed in Table 1. Table 1 also lists the concentrations (in weight percentage) of the various ingredients for all the examples 1 to 4.

In Example 1, a composition as shown in Table 1 was prepared, and did not contain either niacinamide or allantoin.

In Example 2, a composition as in Example 1 was prepared but with 1.0% of niacinamide added.

In Example 3, a composition as in Example 1 was prepared but with 0.2% of allantoin added.

In Example 4, a composition as in Example 1 was prepared but with both 1% niacinamide and 0.2% allantoin being added.

Test Method

The skin creams as prepared were tested on the forearms of 12 women over a course of 5 days with areas marked where the creams were used, and an area was marked where cream was not used. This was conducted as a blind test, where neither the panellists nor the study person was aware of the contents of the cream used. The difference in the skin colour score of the untreated and the treated site gave the skin lightening score, with the more negative number indicating better skin lightening effect.

Table 1 also lists the skin lightening score of the various examples 1 to 4.

TABLE 1

| | Example 1 | Example 2 | Example 3 | Example 4 |
|---|---|---|---|---|
| Ingredients | wt % | wt % | wt % | wt % |
| Niacinamide | — | 1.0 | — | 1.0 |
| Allantoin | — | — | 0.2 | 0.2 |
| Stearic Acid | 18 | 18 | 18 | 18 |
| Silicone oil | 0.5 | 0.5 | 0.5 | 0.5 |
| Sunscreens | 0.14 | 0.14 | 0.14 | 0.14 |
| Preservatives | 0.5 | 0.5 | 0.5 | 0.5 |
| Other additives | 2.05 | 2.05 | 2.05 | 2.05 |
| Water | To 100 | To 100 | To 100 | To 100 |
| Skin lightening score | −0.23 | −0.30 | −0.26 | −0.40 |

The data as per Table 1 indicates that the composition as per the invention (Example 4) gives synergistic skin lightening benefits.

1. A skin lightening composition comprising;
a) 0.05% to 10% by weight of one of more of vitamin B3 compounds or its derivatives thereof;
b) 0.01% to 10% by weight of allantoin; and
c) a cosmetically acceptable vehicle or 15% to 85% by weight detergent active.

2. The skin lightening composition of claim 1 wherein the vitamin B3 compound is selected from niacin, niacinamide, nicotinyl alcohol, or derivatives or salts thereof.

3. The skin lightening composition of claim 1 wherein the vitamin B3 derivative is selected from nicotinic acid esters, nicotinyl alcohol esters of carboxylic acids, niacinamide N-oxide, nicotinyl amino acids and nicotinic acid n-oxide

4. The skin lightening composition of claim 1 wherein the vitamin B3 compound is niacinamide present in 0.2% to 5% by weight.

5. The skin lightening composition of claim 1 wherein allantoin is present at a level of 0.05% to 5% by weight.

6. The skin lightening composition of claim 1 additionally comprising 0.1% to 10% by weight of one or more sunscreens.

7. The skin lightening composition of claim 1 wherein the sunscreen is selected from 2-ethyl hexyl-p-methoxycinnamate, 4,4′-t-butyl methoxydibenzoyl-methane, titanium dioxide or mixtures thereof.

8. The skin lightening composition of claim 1 additionally comprising vitamin B6.

9. The skin lightening composition of claim 1 wherein the composition is a leave on product.

10. The skin lightening composition of claim 1 wherein the composition is in the form of a soap bar.

11. A cosmetic method of lightening skin comprising applying thereto a composition according to claim 1.

12. A cosmetic method according to claim 11 wherein the composition is applied to and left on the skin.

13. A cosmetic method according to claim 11 wherein the composition is applied to the skin and subsequently washed off.
